(19) United States
(12) Patent Application Publication
(10) Pub. No.: US 2008/0046191 A1
(43) Pub. Date: Feb. 21, 2008

(54) System, Process And Software Arrangement For Disease Detection Using Genome Wide Haplotype Maps

(76) Inventors: Bud Mishra, Great Neck, NY (US); Thomas Anantharaman, Poughquag, NY (US)

Correspondence Address: DORSEY & WHITNEY LLP;INTELLECTUAL PROPERTY DEPARTMENT, 250 PARK AVENUE, NEW YORK, NY, 10177, US

(21) Appl. No.: 10/553,618

(22) PCT Filed: Nov. 20, 2003
(86) PCT No.: PCT/US03/37114

§ 371 Date: Aug. 17, 2007

Related U.S. Application Data
(60) Provisional application No. 60/427,903, filed on Nov. 20, 2002.

(51) Int. Cl. G06F 19/00; G01N 33/48

(52) U.S. Cl. 702/20

(57) ABSTRACT

System, process and software arrangement produces high resolution, high accuracy, ordered, genome wide haplotyped maps from single molecule based approximate ordered maps and the location of genes responsible for genetic diseases are determined by performing an association study using a population of genome wide haplotyped maps. This can also be used with Optical Mapping data to assemble a genome wide haplotyped restriction map based on multiple distinguishable restriction enzymes. This invention can also be used with any other single molecule process that can produce approximate ordered physical map from randomly broken DNA pieces of a particular genome.

### CROSS-REFERENCE TO RELATED APPLICATION

The present application claims priority from U.S. Patent Application No. 60/427,903, filed Nov. 20, 2002, the entire disclosure of which incorporated herein by reference.

### FIELD OF THE INVENTION

The present invention relates to systems, process and software arrangements for producing genome wide haplotyped maps. More particularly, the present invention relates to systems, process and software arrangements for producing genome wide haplotyped maps from single molecule based approximate ordered maps and locating genes responsible for genetic diseases.

### BACKGROUND OF THE INVENTION

One of the goals of genomics is to locate genes responsible for genetic diseases. The traditional approaches to locating such genes are generally based on finding single polymorphic genetic markers that are co-inherited with the disease with such regularity that it can be assumed that the single disease-causing gene is located very close to the marker. These approaches are traditionally divided into two classes, Linkage Analysis, as described in Neil J. Risch, “Searching for Genetic Determinants in the New Millennium” Nature, 405, June 2000, the disclosure of which is incorporated herein by reference, and (single marker) Association Studies as described in Thomas G. Schulze and Francis J McMahon, “Genetic Association Mapping at the Crossroads: Which Test and Why? Overview and Practical Guidelines” American Journal of Medical Genetics (Neuropsychiatric Genetics) volume 114, pages 1-11 (2002), the disclosure of which is incorporated herein by reference. Both these conventional approaches typically only track a single marker, and therefore do not work for multi-genic diseases, which are now believed to predominate in all undiscovered disease genes. In addition, both approaches generally use complex statistical process to compensate for spurious correlations that can occur due to population stratification and other unknown and non-random genetic variation across the genetic samples studied, which almost always requires samples from related individuals.

Both of these types of problems could be obviated by using genome wide maps of (polymorphic) genetic markers. If all possible polymorphic genetic markers are available across a large enough set of samples, it is easy to statistically compensate for spurious correlations by randomly sampling large numbers of markers that most likely are not related to the disease of interest. In addition, it is possible to locate all genes involved in multi-genic diseases. The estimate of a statistically sufficient sample size for this problem remains elusive as it depends on the complexity of multi-genic disease with an unknown structure.

The down side is that the cost of genome wide maps of polymorphic markers is very high even in the current post-genomic era. For example, the most common polymorphic marker, a SNP, is expected to cost about 5¢ cents per marker in the near future. However, there are estimated to be 10 million such markers over the entire human genome, and a realistic association study would require at least 1000 samples to be tested for each of such 10 million SNPs. Fortunately, recent results show the presence of significant linkage disequilibrium, as described in David Altshuler et. al., “The Structure of Haplotype Blocks in the Human Genome”, Science, 296, June 2002, the disclosure of which is incorporated herein by reference, suggesting that the human genome can be broken into haplotype blocks of average size of 30 Kb, with all polymorphic markers within a single haplotype block being nearly 100% correlated with each other. In addition each such haplotype block appears to have an average of only 5 alleles (genetic variations). Thus, on average, 3 carefully selected SNPs should be enough to identify all genetic variation within each haplotype block, and hence testing for about 300,000 carefully selected SNPs should be enough to identify all genetic variation in a single DNA sample. Thus, the cost of genome wide maps of polymorphic markers is significantly reduced. One small inconvenience of linkage disequilibrium is that it is not possible to narrow down the location of the diseases causing gene any more closely than identifying the haplotype block in which it is located.

One problem with attempting to exploit linkage disequilibrium is that in order to preserve all genetic information the genome wide map must distinguish the two parental DNA strands in the sample (except, of course, for the Y chromosome), so that the allele of each parental DNA strand of each haplotype block can be uniquely identified. Such a genome wide map is referred to as a haplotype map (or haplotype block map), and would likely be two maps per chromosome, except for the Y chromosome. Unfortunately, the most inexpensive SNP genotyping process, whether using assays or array hybridization, do not track the phasing between neighboring SNPs. For a genotyping process to be able to track phasing between neighboring polymorphic markers, it should ultimately be able to test single DNA fragments containing 2 or more polymorphic markers in a single test or needs to simultaneously test groups of related DNA samples (e.g. trios of father-mother-child) to distinguish the parental alleles which would increase the total cost of the association study, as well as reducing the applicability for patients that do not have parental DNA available for analysis.

### SUMMARY OF THE INVENTION

The present invention uses single molecule maps, such as generated by Optical Mapping, and is generally based on statistically combining single molecule restriction maps of long genomic DNAs of average length of about 1 Mb; such a segment in human typically contain more than 2 heterozygous polymorphic markers. Thus, it is possible according to the present invention to combine this raw optical mapping data into genome wide haplotype restriction maps. In addition to being able to generate genome wide haplotype restriction maps, the exemplary embodiment of the system, process and software arrangement according to the present invention has two additional advantages over SNP based approaches. First, restriction maps can reveal not only SNPs that coincide with the restriction sites, but also other polymorphisms such as micro-insertions and deletions, global rearrangements or hemizygous deletions. Second, since single DNA molecule segments can be mapped using fluorescent microscopy, the exemplary approach is capable of very high throughput (limited primarily by the digital camera throughput) using very little DNA, and having a fraction of the comparable cost for the least expensive SNP approaches. The commercial cost estimated by the end of 2003 is the equivalent of 2 cents per (phased) genetic marker, and such cost is expected to drop by at least another order of magnitude as faster/cheaper computers and digital cameras become available over time.

The raw single molecule map data can consist of approximate restrictions maps of random pieces or segments of genomic DNA with average length of currently about 1-3 Mb. Each approximate map may be derived from a single such segment of uncloned DNA molecule, directly derived from a blood sample. The map is approximate in that it has a number of errors, including sizing errors in the measurement of fragment size or distance between the restriction sites (typically 10% for a 30 Kb fragment for Optical Mapping), missing restriction sites (typically 20% of restriction sites are false negatives), false restriction sites (typically 10% of restriction sites are false positives), and missing small fragments (typically most fragments under 1 Kb are missing). Algorithms to assemble such approximate maps into larger and highly accurate maps using redundant data (50× is typically sufficient) have been used successfully to construct genome wide (non-haplotype) restriction maps of micro-organisms such as E. Coli and P. Falciparum as well as BAC clones of human DNA, as described in Lim A, Dimalanta E T, Potamousis K D, Yen G, Apodoca J, Tao C, Lin J, Qi R., Skiadas J, Ramanathan A, Pema N T, Plunkett G 3rd, Burland V, Mau B, Hacket J, Blattner F R, Anantharaman T S, Mishra B, Schwartz D C. “Shotgun optical maps of the whole Escerichia coli O157:H7 genome”, Genome Research, 11(9): 1584-93, September 2001; Giacalone J, Delobette S, Gibaja V, Ni L, Skiadas Y, Qi R, Edington J, Lai Z, Gebauer D, Zhao H, Anantharaman T, Mishra B, Brown L G, Saxena R, Page D C, Schwartz D C. “Optical mapping of BAC clones from the human Y chromosome DAZ locus,” Genome Research, 10(9): 1421-9, September 2000 and Lai Z, Jing J, Aston C, Clarke V, Apodaca J, Dimalanta E T, Carucci D J, Gardner M J, Mishra B, Anantharaman T S, Paxia S, Hoffman S L, Venter J C, Huff E J; Schwartz D C. “A Shotgun Sequence-Ready Optical Map of the Whole Plasmodium Falciparum Genome,” Nature Genetics, 23 (3): 309-313, November 1999 and bud Mishra and Laxmi Parida, “Partitioning K clones: Inapproximability Results and a Practical Solution to the K-Populations Problem”, RECOMB98 pages 192-201, 1998, the entire disclosures of which are incorporated herein by reference. The algorithms used can be based on Maximum Likelihood scoring using a Bayesian prior, as disclose in Anantharaman T, Mishra B, and Schwartz D C, “Genomics via Optical Mapping II: Ordered Restriction Maps,” Journal of Computational Biology, 4(2):91-118, Summer 1999, the disclosure of which is incorporated herein by reference. Similar to other genomic mapping techniques, these algorithms construct only a single consensus map for each human chromosome pair.

The system, process and software arrangement according to the present invention can use any ordered maps of small pieces of DNA from the Genome, provided the markers are polymorphic and the error rates are within the bounds listed in the claims, e.g., data generated by Optical Mapping. This invention can then be used to construct genome wide haplotype maps from any single molecule mapping data and then applied to large-scale association studies to locate the genes responsible for specific genetic diseases.

Optical Mapping, as described in International Application No. PCT/US01/30426, the entire disclosure is incorporated herein by reference, can be used to generate approximate restrictions maps of pieces of single DNA molecules at very low cost and high throughput. Uncloned DNA (e.g., directly extracted from a blood sample) can be randomly sheered into 1-2 mega base pieces and attached to a suitable substrate, where it is first reacted with the restriction enzyme, then stained with a suitable fluorescent dye. The restriction enzyme cleavage sites show up as breakages in the DNA under fluorescent microscope. Tiled images of the surface may be collected automatically using a fluorescent microscope with a computer controlled x-y-z sample translation stage. The images are analyzed automatically by a computer to detect the bright DNA molecules and to locate the breaks in these molecules corresponding to the restriction enzyme cleavage sites. The approximate size of the distance between restriction sites can be estimated based on the integrated fluorescent intensity relative to that of a standard DNA fragment (typically some small cloned piece of DNA, for example some Lambda Phage Clones) that has been added to the sample. The software arrangement by the computer uses the known length and restriction map of the standard to recognize it in the data. Errors can be introduced by the physical process, such as non-uniform staining, failure of restriction enzyme to cleave, random breakage in the DNA molecule that cannot be distinguished from a cleavage site, and errors in the image processing that may introduce additional cleavage sites (due to non-uniform staining) or miss some cleavage sites that produce very small gaps, or accidentally combine two DNA pieces into a single larger piece. These errors include, e.g., sizing errors in the measurement of fragment size or distance between restriction sites (typically 10% for a 30 Kb fragment), missing restriction sites (typically 20% of restriction sites are false negatives), false restriction sites (typically 10% of restriction sites are false positives), and missing small fragments (typically most fragments under 1 Kb are missing). Optical Mapping relies on redundant data to recover from errors. Approximately 50× redundancy is preferred to assemble genome wide maps and recover from most errors (except for a residual sizing error) with high confidence.

A single restriction map generally detects only a limited number of polymorphic markers, namely those SNPs that coincide with the restriction site and insertions/deletions that are large enough to result in significant changes in the distance between restriction sites. The system, process and software arrangement according to the present invention overcomes this limitation, since even considering SNPs alone, enough coincide with restriction sites, that a small number (2-10) of restriction maps may be sufficient to identify the alleles of most haplotype blocks, and thus contain at least as much information as about 300,000 (phased) SNPs.

The exemplary embodiment of the present invention relates to systems, process and software arrangements for producing genome wide haplotyped maps. More particularly, the present invention relates to systems, process and software arrangements for producing genome wide haplotyped maps from single molecule based approximate ordered maps and locating genes responsible for genetic diseases.

Other and further objects, features and advantages of the present invention will be readily apparent to those skilled in the art upon a reading of the description of preferred embodiments which follows.

### DETAILED DESCRIPTION

The present invention relates to systems, process and software arrangements for producing genome wide haplotyped maps. More particularly, the present invention relates to systems, process and software arrangements for producing genome wide haplotyped maps from single molecule based approximate ordered maps and locating genes responsible for genetic diseases.

The prevalence of SNPs that coincide with restriction sites can be estimated quite reliably by examining the set of known SNPs and for each possible restriction enzyme determining if there is a restriction site at the SNP location that would not cut for one of the SNP variants. Such a SNPs site can be referred to as a polymorphic restriction site relative to the restriction enzyme considered. The number of such polymorphic restriction sites for each of the 269 distinct restriction enzymes is shown in Table 1 for a selected subset of restriction enzymes (under the column “Poly Site”). Additional columns adjust the raw number to account for the unknown SNPs that have not yet been detected, but would show up in a restriction map. The last column of the tables assumes that the total number of SNPs is 10 million and is linearly extrapolated from the number of polymorphic restriction sites and known SNPs (1.28 million). In addition, some of the polymorphic restriction sites may not be detected by Optical mapping since they are too close to another restriction site to be resolved by Optical mapping. It can be assumed that any polymorphic restriction site within 400 base pairs of another restriction site should not be detected and estimated the fraction of restriction sites that may be lost on average by examining the distribution of restriction fragment sizes from the sequence of chromosome 21 published by NIH (as shown in column “Miss-rate” in Table 1) and extrapolating this rate to the entire human genome. The last column of Table 1 reflects such adjustment. Optical Mapping generally works on human DNA if a methylation insensitive restriction enzyme is used. There are 8 such known restriction enzymes, which are shown in Table 1 marked with an asterix under the “Methyl” column along with a small selection of other restriction enzymes. The ability to use any particular restriction enzyme can be further restricted by the smallest fragment size the Optical mapping can size reliably. This currently may limit Optical Mapping to restriction enzymes that produce average fragment sizes of 15 Kb or more, and limit the use of the last two restriction enzymes in Table 1 (e.g., PacI and SwaI). However, the sizing accuracy would improve sufficiently to allow maps with average fragment size of about 2.0 Kb to be generated. This would allow the use of any of the last six methylation insensitive restriction enzymes shown in Table 1. One shows how much overlap there is between the information provided by different restriction enzymes. Preferably, if there is no overlap, it is possible to simply add the numbers in the last column of Table 1 to estimate the total number of SNPs that can be detected by using multiple restriction enzymes. In this case, the six methylation insensitive restriction enzymes that are usable by Optical Mapping can detect approximately 200,000 SNPs.

TABLE 1
restriction sites coinciding with SNPs

| | | | Fragsize | | Sites/10M |
|---|---|---|---|---|---|
| Methyl. | Pattern | Poly Sites | (Kb) | Miss-rate | SNPs |
| * | AATT | 48,912 | 0.789 | 0.507 | 94,552 |
| * | TTAA | 41,937 | 0.827 | 0.393 | 92,865 |
| | . . . | | | | |
| | YACGTR | 12,925 | 3.983 | 0.007 | 88,666 |
| | ACRYGT | 9,216 | 2.912 | 0.016 | 57,572 |
| * | TTTAAA | 10,548 | 2.055 | 0.040 | 57,025 |
| * | AATATT | 8,932 | 3.107 | 0.019 | 53,989 |
| | ACGGA | 7,901 | 2.406 | 0.021 | 50,028 |
| | GTMKAC | 8,345 | 3.817 | 0.008 | 49,096 |
| | . . . | | | | |
| * | TTATAA | 6,883 | 4.102 | 0.009 | 45,017 |
| | ... | | | | |
| * | ATTAAT | 5,448 | 4.467 | 0.008 | 34,936 |
| | . . . | | | | |
| * | ATTTAAAT | 980 | 26.648 | 0.000 | 7,520 |
| | . . . | | | | |
| * | TTAATTAA | 773 | 33.504 | 0.000 | 5,509 |

In one exemplary embodiment of the present invention, algorithms can be used to assemble genome wide haplotype maps from Optical mapping data. The map assembly algorithms used to assemble non-haplotype maps from Optical Mapping data may be based on Bayesian/Maximum-Likelihood estimation, as disclosed in Anantharaman T S, Mishra B, and Schwartz D. C., “Genomics via Optical Mapping III: Contiging Genomic DNA and variations.” ISMB99, 7: 18-27, August 1999, the disclosure of which is incorporated herein by reference. The systems, processes and software arrangements of the present invention for assembling haplotype maps from, e.g., Optical Mapping data, extend these algorithms to handle a mixture hypothesis of pairs of maps for each chromosome, corresponding to the correct restriction map of the two parental chromosomes, and each single-molecule Optical Map can be assumed to have been derived from one of these two hypothesis maps at random. For the sake of simplicity, it can be assumed that all data is derived from a single chromosome so that only one pair of hypothesis maps, e.g., H1 and H2 are used. The general case may be a trivial extension of this special case. It is then possible to use a probabilistic model of the errors in the Optical Maps to derive conditional probability density expression ƒ(D|H₁) and ƒ(D|H₂) that any particular approximate restriction map D is derived with errors, and some suitable breakage from correct chromosome maps H1 and H2. The goal is to compare different possible H1 and H2 to find the best ones. Hence, it is possible to apply Bayes rule, Equation (0.1) (with M=number of approximate restriction maps in the input data):

ƒ(H₁,H₂|D₁ . . . D_{M})∝ƒ(H₁,H₂)ƒ(D₁ . . . D_{M}H₁,H₂) (0.1)

The first term on the right side is the prior probability of any hypothesized chromosome maps H1 and H2. Generally, no prior information is available except that the average restriction fragment size is typically approximately known, and it is known that H1 and H2 will be very similar. Polymorphic restriction sites are typically rare around 4% (see last column in Table 1), but can range from 27% (Bpu1831I) to 1.8% (for SdiI) of all restriction sites, depending on the restriction enzyme involved, and can be estimated quite reliably for the restriction enzyme used from the full version of Table 1. For restriction fragment length polymorphisms (RFLPs) there is difficulty estimating how frequently they occur, but it is always possible to estimate a probability (say 4%), and iterate the process if the final maps H1 and H2 that maximize the probability density do not confirm this value. After the first haplotype map with a particular restriction enzyme has been constructed, reliable estimates should carry over to additional maps. Thus, establishing the expression for the prior term is usually possible, and can be further simplified to include only the low prior probability of polymorphic restriction sites or restriction fragment lengths with negligible loss in accuracy.

For the conditional probability term, it can be assumed that each approximate restriction map (data input) is a statistically independent sample from the genome and that the associated mapping errors are independent, and that molecules were derived from either parental chromosome with equal likelihood. Hence, the following expressions can be obtained:

$$f ( {D}_{1} … {D}_{M} \vert {H}_{1} , {H}_{2} ) = {\prod }_{j = 1}^{M} ( f ( {D}_{j} \vert {H}_{1} ) + f ( {D}_{j} \vert {H}_{2} ) ) / 2 \qquad ( 0.2 )$$

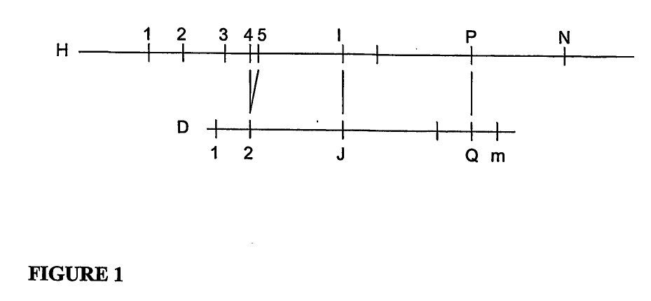

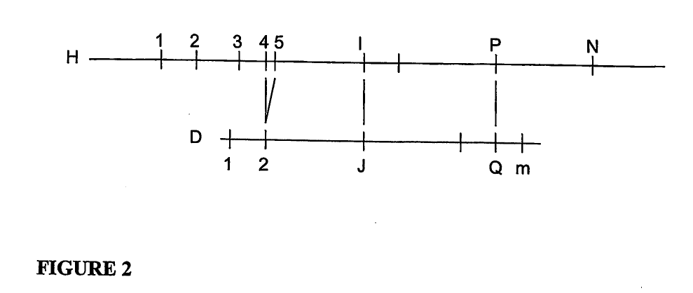

Thus, the conditional probability terms are reduced to combinations of the non-haplotype case ƒ(D|H) involving just one hypothesized map at a time. This conditional term can be provided as a summation over all possible (e.g., mutually exclusive) alignments between the particular D and H, and for each alignment the probability density can be based on an enumeration of the map errors implied by the alignment. In order to obtain a reasonably fast evaluation of the probability densities summed over all alignments is the use of a dynamic programming recurrence equation, which is equivalent to factoring out the common sub-expressions of the probability densities across the different alignments. First, a single arbitrary alignment between a particular D and H should be considered. For the sake of convenience, the following discussion drops the subscript j from D and m). The data map D can be described by a vector of locations of restriction sites D[J=0 . . . m+1], where for convenience the first entry D[0] is 0 and the last entry D[m+1] is the total size of the map. For notational convenience, it is possible to also refer to the entries of this array as D_{J}, J=0 . . . m+1 which should not be confused with the distinct data maps Dⱼ, j=1 . . . M referred to previously. Similarly, the hypothesis map H can be described by a vector H[I=0 . . . N+1] also denoted as H_{I},I=0 . . . N+1. An arbitrary alignment can be provided as a list of pairs of restriction sites from H and D that describe which restriction site from H is aligned with which restriction site from D. According to the example shown in FIG. 1, the alignment consists of 4 aligned pairs (4,2) (5,2) (I,J) and (P,Q). All restriction sites in H or D need be aligned. For example, between aligned pairs (I, J) and (P, Q), there is one misaligned site on H and D each, corresponding to a missing site (false-negative) and extra-site (false-positive) in D. In this alignment, a true small fragment between sites 4 and 5 in H are missing from D, which is shown by aligning both sites 4 and 5 in H with the same site 2 in D. If two or more consecutive fragments in H are missing in D, this can be described by aligning all sites for the missing fragments in H with the same site in D (rather than showing only the outermost of this set of consecutive sites in H aligned with D, for example). This convention provides that for each missing fragment two consecutive sites in H (those flanking the missing fragment) can be aligned with the site in D in which the fragment is presumed missing.

The expression for the conditional probability density of any alignment such as this can be provided as the product of a term corresponding to the region of alignment between each pair of aligned sites, plus one term for the unaligned region at each end of the alignment. For an aligned region that is not a missing fragment (e.g. (I, J) and (P, Q), such that P>I and Q>J), this probability density can be denoted by a function of the form FA_{I,J,P,Q}, which may depend on the specific errors in the corresponding region of the alignment between D and H. Similarly for an aligned region that corresponds to a consecutive number of missing fragments, the probability density may be denoted by a function FM_{I,P} (e.g. (I,J) and (I+1,J) can correspond to FM_{I,I+1}). For the probability density of the unaligned portion on the left and right end of each alignment, UR_{I,J} can be used on the right end if (I, J) is the rightmost aligned pair, and UL_{I,J} on the left end if (I, J) is the leftmost aligned pair.

Their exact form does not affect the complexity of the system, process and software arrangement according to the present invention, as long as they can be evaluated in constant time. The form of these functions for a good Optical Mapping data model is shown in example 1 in equations (0.7)(0.8) and (0.9).

The probability density of a particular alignment is the product of each of the terms FA_{I,J,P,Q}, PM_{I}, UL_{I,J}, UR_{I,J} that apply to that alignment. The probability density of any alignment can be separated into the product of those terms on either side of any particular alignment pair (I, J). This forms the basis of a two-dimensional recurrence using an array AR_{I,J}, where I=1 . . . N, J=0 . . . m+1. AR_{I,J} represents the sum of the probability densities of all those alignments between the part of H to the right of site I, and the part of D to the right of site J, for which (I, J) is the leftmost aligned pair. Thus, it is possible to derive the recurrence for AR_{I,J} in Equation (0.3).

$$A {R}_{I , J} = U {R}_{I , J} + ( I \geq N ? 0 \text{:} 1 ) F {M}_{I , I + 1} A {R}_{I + 1 , J} + {\sum }_{P = I + 1}^{N} {\sum }_{Q = J + 1}^{m + 1} A {R}_{P , Q} F {A}_{I , J , P , Q} \qquad ( 0.3 )$$

This array can then be used to compute the total probability density by summing over every possible leftmost alignment pair (I, J) as shown in Equation (0.4).

$$f ( D \vert H ) = {\sum }_{I = 1}^{N} {\sum }_{J = 0}^{m + 1} A {R}_{I , J} U {L}_{I , J} \qquad ( 0.4 )$$

Equations (0.3)(0.4) are able to sum up all of the alignments in time proportional to mⱼ²N², where mⱼ is the number of restriction sites in Dⱼ and N is the number of restriction sites in H. If an acceptable a good approximate location of the best alignment between D and H is known, which is possible if the conditional density has been previously evaluated for a similar H or with the help of geometric hashing algorithms, a constant width band of the recurrence array AR_{I,J} should be evaluated which can be performed in time proportional to 2mⱼΔ³, where Δ is the number of restriction sites representing the width of the band. A fixed value of Δ=8 works well for error rates typical in Optical Mapping data.

The computationally expensive part is the search over possible correct maps H1 and H2. First, assuming that both H1 and H2 is very similar, and a single hypothesis H that best matches all data can be reached for. This first stage is similar to the case of non-haplotype map assembly. Then the maps can be heuristically and quickly assembled into larger contigs using a similar and approximate dynamic programming scheme to obtain the best alignment between any two approximate maps D. If this alignment is good enough, the maps can be combined into a larger map (contig map) by averaging the two maps in their overlap region. This heuristic stage relies on geometric hashing to quickly identify the maps that overlap, and the complexity of this stage can be determined by the geometric hashing and is estimated to be approximately O (M_{D}^{4/3}) where

$${M}_{D} ≡ {\sum }_{j = 1}^{M} {m}_{j}$$

is the total number of fragments in the Optical Mapping data. Geometric hashing can have sub-quadratic complexity in the worst case and the complexity may be as good as linear. The actual time for this state of computation is usually small compared to the time for the remaining search over possible H1 and H2, unless the genome being used is much larger than the human genome. The resulting contig maps can be used as a basis for an initial hypothesis H, which should then be refined by trying to add or delete restriction sites and by adjusting the distance between restriction sites by doing a gradient optimization of the probability density of all maps for each fragment size. The first two derivatives of ƒ(D|H) with respect to any single fragment size can be computed by a recurrence similar to AR_{I,J}, by taking the derivatives of the recurrence equations applying the normal chain rule. Outlined below is an algorithm that can compute the derivative for all fragment sizes in a single step only 2-3 times as expensive as doing so for a single fragment size.

This initial search stage, which constructs a genotype map H, is then followed by an additional search in which H1 and H2, initially the same as the best H, are gradually modified by attempting to introduce a restriction site polymorphism at each site in H1 or H2 (and also at locations between them) as well as restriction fragment length polymorphisms (RFLPs) for each fragment in H1 or H2 and evaluating the complete probability density using Equation (0.1). Attempting each new restriction site polymorphism involves modifying H1 or H2 by adding or deleting a restriction site from H1 (or H2) only, while attempting an RFLP involves modifying the same interval in H1 and H2 by adding some delta to H1 and subtracting the same delta from H2. In each case, 2 possible orientations of each polymorphism are possibly, reversing the use of H1 and H2 above. Both orientations should be tested and the better scoring orientations selected, except when adding the first polymorphism to H1 and H2. In this manner the correct phasing of neighboring polymorphisms can be detected in a natural manner whenever possible. If the data cannot allow the phasing to be determined because there may be insufficient or no data molecules spanning both polymorphisms, both phases (orientations) can have the same score . This fact is also recorded since it marks a break in the phasing of polymorphisms, and the interval between such breaks can be referred to as a “phase contig.” RFLP polymorphisms are more expensive to score, since in addition to the orientation (whether H1 or H2 has the bigger fragment) estimates are generally made regarding the value of delta (the amount of the fragment size difference for H1 and H2), which can involve some form of trial and error procedure.

By testing a preliminary implementation of the above algorithm on simulated data, a purely greedy addition of polymorphisms to H1 and H2 can get lodged in local maxima when two or more actual polymorphisms are in a close vicinity. For example, if the true H1 has a 10 Kb fragment followed by a 1 Kb fragment while the true H2 has an 11 Kb fragment in the same location, the correct solution is to add a restriction site polymorphisms to the initial contig map at the right end of the 10-11 Kb fragment. However, given the possibility of sizing errors and missing small fragments of 1 Kb, it is also possible to score this as a RFLP (the 10 Kb vs. 11 Kb) and the 1 Kb fragment being missing in half the data. By attempting both cases before committing to a change in H1 and H2, the restriction site polymorphism can score slightly higher than the RFLP. This can be implemented by using a heuristic look ahead distance of a certain number of restriction sites, and scoring all alternate possible polymorphisms within this distance of the best scoring one, before committing the best scoring polymorphism in H1 and H2. In general, it is possible to score all possible pairs (or triples) of polymorphisms in a local region, which would increase the search cost.

Simple heuristics can be used to significantly accelerate the evaluation of Equation (0.1). First H1 and H2 are typically modified in a single location at a time. Data maps are typically only 1-2 Mega bases, while a complete chromosome map represented by H1 or H2 can be much larger. If a data map Dⱼ did not previously overlap H1 or H2 anywhere near the location being modified, the conditional probability density terms ƒ(Dⱼ|Hᵢ), can be reused for that data map from the last time it was evaluated. This effectively makes the cost of re-evaluating Equation (0.1) for a local change proportional to the coverage depth times mⱼ the number of restriction sites per map, rather than M_{D} the total number of restriction sites in all data maps. Since all restriction sites should be considered 2-3 times until it is assured that no further improvements to H1 and H2 are possible, this makes the total cost of the search for the best H1 and H2 proportional to (M_{D}|C)mC=M_{D}m, where m is the average number of sites per data map D, and C is the coverage depth. Since this usually dominates the O (M_{D}^{4/3}) cost for the initial map H, the total cost remains roughly proportional to the total number of restriction sites in the data.

Second, for the case of restriction site polymorphism, it is possible to accelerate the program by another factor of 2 by avoiding evaluating both possible orientations separately. Referring to Equation (0.2), i.e., that each hypothesis H1 or H2 can occur in just two version for any particular restriction site; either the restriction site is present or it is absent. For example, if previously both H1 and H2 have a restriction site present Equation (0.2) is reevaluated first with the restriction site deleted from just H1, next with the restriction site deleted from just H2. Since it is possible to remember the previous values of these terms (with the restriction site present in H1 and H2), these terms can be recomputed and recoded with the restriction site absent in both H1 and H2 and then perform the inexpensive averaging operations twice by combining the appropriate probability density terms already computed. It is also possible to evaluate the case where neither H1 nor H2 have a restriction site at almost no extra cost, which can be the best option as a result of other changes in H1 and H2 nearby.

Both of these simple heuristics provide significant acceleration, while the resulting program can currently take about 2 hours per Mega base to search over the possible space of H1 and H2. In the next section describes improvements to these algorithms in order to provide the acceleration of 20-140× or more, in addition to ways to parallelize the algorithms for a 16 or 96 processor Linux cluster.

In a further embodiment of the present invention, a system, process and software arrangement to accelerate a single processor performance of the haplotype map assembly can be provided. An algorithm used by the system process and software arrangement for assembling the map and locating and phasing all polymorphisms in time proportional to O(M_{D}^{4/3}+M_{D}m) where M_{D} is the total number of fragments in all input maps and m is the average number of fragments per input map has been described above. The second term dominates the time complexity for a human genome, and is due to the evaluation of the probability density repeatedly for different assumed parental chromosome maps H1 and H2. An algorithm is described that will drop the complexity of the second term to O(M_{D}). However, this algorithm has a constant overhead that we estimate at about 4-6×. Hence, the potential speed up is likely to be about m/4 to m/6, which with an average fragment size of 15 kB and an average molecule size of 2 MB is about 20-30×. For an average fragment size of 2 kB, the acceleration is even greater at over 150×, and the time now remains proportional to the total number of input fragments and hence the total time increases by a factor of 7.5×.

This exemplary embodiment provides fast way to re-evaluate ƒ(D|H) when H has been changed locally in just one place in any of the following ways:
1. Delete one of the existing restriction sites in H.
2. Add a new restriction site at a specified location in H.
3. Increase or decrease one of the fragments (restriction site intervals) in H by a specified amount.
4. The first and second derivative of ƒ(D|H) relative to any fragment size in H.

In all of the above cases, e.g., the cost of all such evaluations of ƒ(D|H) (or its derivatives) for all restriction sites spanning the molecule D can be done in just 2-3 times the time it previously took to do just one such evaluation at one restriction site. This will allow the evaluation of Equations (0.1) (0.2) for all possible changes over a window of 2m restriction sites in time that is just 4-6 times greater than the cost for testing a single change at a single restriction site previously. The extra factor of 2 is due to the fact that the number of molecules D for which ƒ(D|H) is recomputed roughly double.

The first step is to compute a new recurrence array AL_{I,J} which represents the sum of the probability densities of all those alignments between the part of H to the left of site I and the part of D to the left of site J, for which (I, J) is the rightmost aligned pair. As previously discussed the corresponding recurrence equation can be derived as follows:

$$A {L}_{I , J} = U {L}_{I , J} + ( I \leq 0 ? 0 \text{:} 1 ) F {M}_{I - 1 , I} A {L}_{I - 1 , J} + {\sum }_{P = 1}^{I - 1} {\sum }_{Q = 0}^{J - 1} A {L}_{P , Q} F {A}_{P , Q , I , J} \qquad ( 0.5 )$$

This array is preferably the mirror image of AR_{I,J}, this recurrence array can be used to compute ƒ(D|H) using an Equation similar to Equation (0.4). However, one exemplary reason to compute both AR_{I,J} and AL_{I,J} is that if H is changed locally near some restriction site H_{K}, this will not change AR_{I,J} for I<K or AL_{I,J} for I>K. It is possible to use mainly the parts of AR_{I,J} and AL_{I,J} that didn't change to compute ƒ(D|H). Then, the additional cost can be limited to re-computing the parts of AR_{I,J} and AL_{I,J} near I=K. In addition, some of this cost of re-computing can be amortized over different values of K, if the effect of local changes at consecutive restriction sites H_{K} is simultaneously checked. To express ƒ(D|H) in terms of both AR_{I,J} and AL_{I,J} so that those recurrence terms that do not change if we change H near H_{K} are used, the following formulations are used:

$$\begin{matrix}{f}_{K} ( D \vert H ) = P r ( Alignments with righmost aligned I \leq K ) + \\ P r ( Alignments with leftmost aligned I > K ) + \\ P r ( Alignments with a fragment spanning \\ [ {H}_{K} , {H}_{K + 1} ] ) \\ = {\sum }_{I = 1}^{K} {\sum }_{J = 0}^{m + 1} A {L}_{I , J} U {R}_{I , J} + {\sum }_{I = K + 1}^{N} {\sum }_{J = 0}^{m + 1} A {R}_{I , J} U {L}_{I , J} + \\ {\sum }_{J = 0}^{m + 1} \{ \begin{matrix}( K < N ? 1 \text{:} 0 ) A {L}_{K , J} F {M}_{K , K + 1} A {R}_{K + 1} + \\ {\sum }_{I = 1}^{K} {\sum }_{P = K + 1}^{N} {\sum }_{Q = J + 1}^{M + 1} A {L}_{I , J} F {A}_{I , J , P , Q} A {R}_{P , Q}\end{matrix} \}\end{matrix} \qquad ( 0.6 )$$

All instances AR_{I,J} and AL_{I,J} used in Equation (0.6) remain unchanged if the interval H_{K} . . . H_{K+1} in H is changed. Only the non-recurrence terms FA_{I,J,P,Q}, FM_{K,K+1}, UR_{I,J}UL_{I,J} change, and the modified forms of these terms can be computed in approximately constant time.

The exemplary algorithms for each of the 4 cases are described below. To summarize, the computation cost in each of these 4 cases turns out to be:
1. To delete a restriction site from H: Total cost 6mΔ³ for m restriction sites.
2. To change the size of a restriction fragment in H: Total cost 6mΔ³ for changing each of m+1 restriction fragments by the same increment Δ_{H}.
3. To add a restriction site at any point in H: Total cost 6mΔ³+4TΔ⁴ to add one restriction site at T arbitrary locations. Note that to add one restriction site within each fragment (T=m), the total cost is about Δ times more expensive than for the previous 2 cases, since it is not possible to amortize the cost associated with the unique location of each new restriction site.
4. To compute the first two derivatives of ƒ(D|H) relative to each of m+1 fragment sizes: Total cost 8mΔ³ (slightly higher than for first two cases since we have to compute 2 derivatives).

The 2^{nd} case (i.e., changing the size of a restriction fragment), the result is limited to the case when each fragment is changed by the same amount Δ_{H}, otherwise the computation cost is 4mΔ³+4TmΔ²+2TΔ⁴. A possible strategy for finding RFLPs is to first check each fragment using a standard small value of Δ_{H} and −Δ_{H} to check if an RFLP exists. Most fragments do not exhibit any RFLP. For the small number that do, a search can be performed for the optimal Δ_{H} value, using T different Δ_{H}, values over all fragments exhibiting an RFLP may have a computation cost of 4mΔ³+4TmΔ²+2TΔ⁴ if it is started from scratch or a cost of just 4TmΔ³+2TΔ⁴ if the arrays AL_{I,J} and AR_{I,J} are saved for each data molecule from the first phase. For example, if 2 fragments are polymorphic it is possible to iterate 2-3 times with T=20 (10 Δ_{H} values per fragment), thereby reducing the uncertainty in the optimal value of Δ_{H} by a factor of 10 in each such iteration.

In the 4^{th} case computing the two derivatives for each fragment may not be enough. In particular, all fragment sizes should be updated using some approximation to Newton's process, and iterate this a few times (4-10 typically) to insure convergence. Since the diagonal of the Jacobian (2^{nd} Gradient) is computed, the result may be unstable and suitable step size scaling may be preferable to insure convergence. It is also possible to compute a few off diagonal terms of the Jacobian (e.g., the first off diagonal terms resulting in a tri-diagonal Jacobian matrix), if this will accelerate convergence.

The 3^{rd} case prefers to use a systematic way to decide where a new restriction site should be added. Possible strategies may be to attempt 3-5 uniformly spaced locations inside each existing fragment OR every location for which a data molecule currently has a misaligned restriction site. It may be difficult to pick optimal locations in this manner, and therefore may miss the true location, unless an improvement is observed in the value of the total probability density and subsequently optimize the location by optimizing fragment sizes (4^{th} case). In still a further embodiment, a 5^{th} type of local modification to H is provided that is combination of the 3^{rd} and 4^{th} cases. For each proposed new restriction site the new probability density can be computed as well as its first two derivatives relative to the location of the new restriction site, and then use a quadratic extrapolation of the probability density to score the new restriction site.

In still a further embodiment of the present invention, the above described algorithms can apply to each molecule D independently, and they may be executed on a parallel Linux cluster by having each processor work on a separate molecule. It is preferable that each processor's workload is as balanced as possible. Since not all molecules would be of equal size, it may not be possible to obtain exact results. However, since it is possible to obtain a good estimate of the computation time as a function of the molecule size (m in the previous section), known bin-packing heuristics can be used to divide the set of molecules into 16 (or 96) groups that have similar total computation cost. For large data sets (300,000 molecules will be typical for a human genome at 50× coverage), the load balancing can be better than 95%. The bandwidth used between processors can be quite low since the final probabilities for each molecule D and possible local changes to map H are communicated to the master processor responsible for deciding how to modify H.

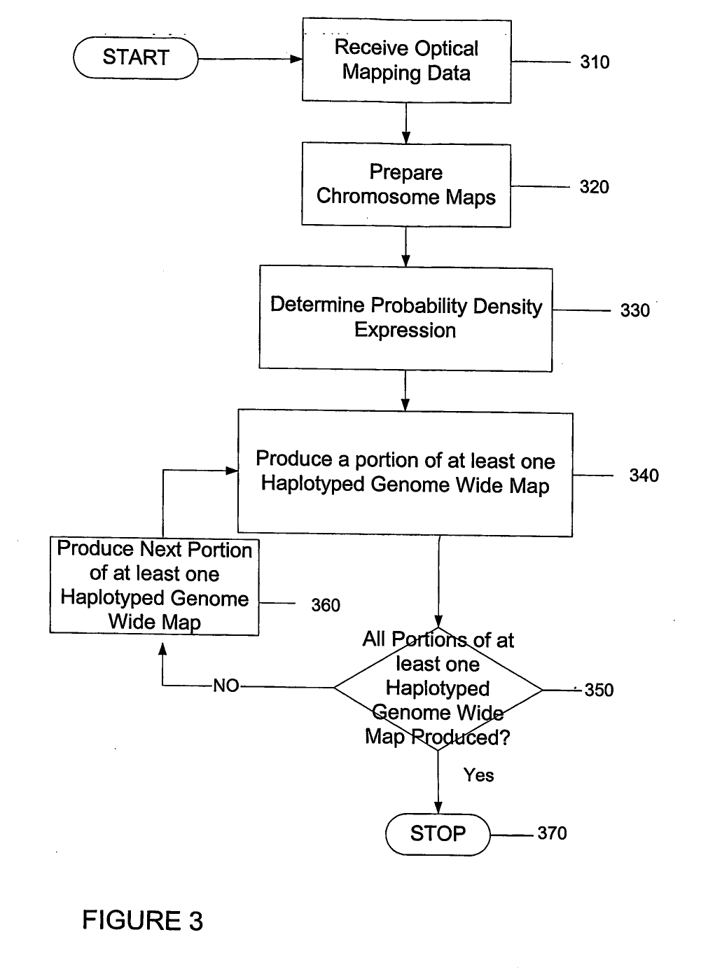

FIG. 3 shows an exemplary flow chart of an exemplary embodiment of the process according to the present invention for producing at least one haplotyped genome wide map. This process can be performed by a processing device, such as for example a computer that includes a microprocessor. The processing device receives data 310, which can be, for example, Optical Mapping data. Then, in step 320, the processing device prepares chromosome maps associated with at least one chromosome. In step 330, a conditional probability density expression can be determined using the Optical Mapping data. Then, in step 340, a portion of at least one haplotyped genome wide map may be produced. In step 350, the processing device determines whether all portions of the at least one haplotyped genome wide map have been produced. If not, in step 360, a next portion of at least one haplotyped genome wide map can be produced. If all portions have been produced, in step 370, the process stops.

To facilitate a further understanding of the present invention, the following example of some of the preferred embodiments are provided. In no way do such example be read to limit the scope of the invention.

### EXAMPLE 1

According to a process according to one exemplary embodiment of the present invention will now be described, an alignment probability expressions is provided that correspond to a good error model for Optical Mapping data:

$$\begin{matrix}F {A}_{I , J , P , Q} ≡ {{λ}^{Q - J - 1} ( 1 - {P}_{d} )}^{P - I - 1} {P}_{d} G ( {D}_{Q} - {D}_{J} , {H}_{P} - {H}_{I} ) ( 1 - {P}_{v}^{{H}_{P} - {H}_{I}} ) & ( 0.7 ) \\ F {M}_{I , P} ≡ {P}_{v}^{{H}_{P} - {H}_{I}} & ( 0.8 ) \\ U {R}_{I , J} ≡ \{ \begin{matrix}{\sum }_{P = I + 1}^{N + 1} F {R}_{I , J , P , P - 1} , If J \leq m \\ {P}_{v}^{{H}_{N + 1} - {H}_{N}} + {R}_{e} ( {P}_{v}^{{H}_{N + 1} - {H}_{N}} - 1 ) / log {P}_{v} , If I = N and J = m + 1 \\ 0 , otherwise\end{matrix} & ( 0.9 ) \\ U {L}_{I , J} ≡ \{ \begin{matrix}{\sum }_{P = 0}^{I - 1} F {L}_{I , J , P , P + 1} , If J > 0 \\ {P}_{v}^{{H}_{1}} + {R}_{e} ( {P}_{v}^{{H}_{1}} - 1 ) / log {P}_{v ,} If I = 1 and J = 0 \\ 0 , otherwise\end{matrix} & \\ Where , & \\ F {R}_{I , J , P , Q} ≡ {{λ}^{m - J} ( 1 - {P}_{d} )}^{P - I - 1} ( 1 - {P}_{v}^{{H}_{p} - {H}_{I}} ) ( \begin{matrix}{R}_{e} {G}_{E} ( {D}_{m + 1} - {D}_{J ,} {H}_{P} - {H}_{I} , {H}_{P} - {H}_{Q} ) + \\ ( P > N ? 1 \text{:} 0 ) G ( {D}_{m + 1} - {D}_{J} , {H}_{N + 1} - {H}_{I} )\end{matrix} ) & \\ F {L}_{I , J , P , Q} ≡ {{λ}^{J - 1} ( 1 - {P}_{d} )}^{I - P - 1} ( 1 - {P}_{v}^{{H}_{I} - {H}_{P}} ) ( {R}_{e} {G}_{E} \begin{matrix}( {D}_{J} , {H}_{I} - {H}_{P} , {H}_{Q} - {H}_{P} ) + \\ ( P = 0 ? 1 \text{:} 0 ) G ( {D}_{J} , {H}_{I} )\end{matrix} ) & \\ G ( d , h ) ≡ \frac{{e}^{- {( d - h )}^{2} / 2 {σ}^{2} h}}{\sqrt{2 π {σ}^{2} h}} & \\ {G}_{E} ( d , h , b ) ≅ \frac{1}{2} \{ erf ( \frac{d - h + b}{σ \sqrt{2 max ( h - b , min ( d , h ) )}} ) + erf ( \frac{h - d}{σ \sqrt{2 max ( h - b , min ( d , h ) )}} ) \} & \end{matrix}$$

Where P_{d} is the digest rate, and hence (1−P_{d}) is the missing restriction site rate, λ is the false-positive site rate (sites per Mega base for example), σ²h is the Gaussian sizing error variance for a fragment of size h, and Pᵥ is the probability that a fragment of unit size will be missing in the data, and Rₑ is the breakage rate of the original DNA (the inverse of the average size of the DNA maps D). A C-style notation (condition?1:0) is used before a term that should be present if condition is true.

Although it does not appear that UR_{I,J} or UL_{I,J} can be computed in constant time, and likely 3-7 of the terms FR_{I,J,P,Q} or FL_{I,J,P,Q} (which can each be computed in constant time) are significant, these significant terms are stable and can be determined during an initial pass, and updated periodically as H1/H2 change. Equation (0.9) is provided under the assumption that each end of H is the end of a chromosome: the equations are likely simpler if H is an incomplete chromosome.

Next a detailed description of processes for handling each of the four types of local modifications to the true map hypothesis H are described. In particular,
1. Delete an existing restriction site from H.
2. Add a new restriction site at a specified location in H.
3. Increase or decrease one of the fragments (restriction site intervals) in H by a specified amount.
4. The first and second derivative of ƒ(D|H) relative to any fragment in H.

First, described below is a way to re-compute ƒ(D|H), while deleting one restriction site H_{K} from H at a time for all possible K (1≦K≦N).

The first step is to derive an equation for ƒ(D|H) that uses only those parts of AR_{I,J} and AL_{I,J} that will not change when H_{K} is deleted, while excluding the probability for alignments that align with H_{K}:

$$\begin{matrix}{f}_{κ} ( D \vert H ) = P r ( Alignments with righmost aligned I < K ) + \\ P r ( Alignments with leftmost aligned I > K ) + \\ P r ( Alignments with a fragment spanning \\ [ {H}_{K - 1} , {H}_{K + 1} ] ) \\ = {\sum }_{I = 1}^{K - 1} {\sum }_{J = 0}^{m + 1} A {L}_{I , J} U {R}_{I , J} + {\sum }_{I = K + 1}^{N} {\sum }_{J = 0}^{m + 1} A {R}_{I , J} U {L}_{I , J} + \\ {\sum }_{J = 0}^{m + 1} \{ \begin{matrix}( K < N ? 1 \text{:} 0 ) A {L}_{K - 1 , J} F {M}_{K - 1 , K + 1} A {R}_{K + 1} + \\ {\sum }_{I = 1}^{K - 1} {\sum }_{P = K + 1}^{N} {\sum }_{Q = J + 1}^{M + 1} A {L}_{I , J} F {A}_{I , J , P , Q} A {R}_{P , Q}\end{matrix} \}\end{matrix} \qquad ( 0.10 )$$

In Equation (0.10), none of the terms AR_{I,J} or AL_{I,J} change if the restriction site H_{K} is removed from H. However, the terms FA_{I,J,P,Q} and UR_{I,J} and UL_{I,J} change if H_{K} is deleted. Referring to Equations (0.7)(0.8)(0.9), the change to FA_{I,J,P,Q} is simple, and involves a dropped term of (1−P_{d}). To see the effect on UR_{I,J} and UL_{I,J} these are expanded in terms of FR_{I,J,P,Q} and FL_{I,J,P,Q} according to Equation (0.9) and simplified to obtain:

$${f}_{K} ( D \vert H ) = {\sum }_{I = 1}^{K - 1} {\sum }_{J = 0}^{m} A {L}_{I , J} {\sum }_{P = I + 1}^{N + 1} F {R}_{I , J , P , P - 1} + {\sum }_{I = K + 1}^{N} {\sum }_{J = 1}^{m + 1} A {R}_{I , J} {\sum }_{P = 0}^{I - 1} F {L}_{I , J , P , P + 1} + {\sum }_{J = 0}^{m + 1} \{ \begin{matrix}( K < N ? 1 \text{:} 0 ) A {L}_{K - 1 , J} F {M}_{K - 1 , K + 1} A {R}_{K + 1 , J} + \\ {\sum }_{I = 1}^{K - 1} {\sum }_{P = K + 1}^{N} {\sum }_{Q = J + 1}^{m + 1} A {L}_{I , J} F {A}_{I , J , P , Q} A {R}_{I , J}\end{matrix} \} \qquad ( 0.11 )$$

Equation (0.11) can then be modified to reflect the deletion of H_{K} from H and corresponding changes in FA_{I,J,P,Q}, FR_{I,J,P,Q} and FL_{I,J,P,Q} to obtain the following:

$$\begin{matrix}\begin{matrix}f ( D \vert H - {H}_{K} ) = {\sum }_{I = 1}^{K - 1} {\sum }_{J = 0}^{m} A {L}_{I , J} {\sum }_{P = I + 1}^{N + 1} F R {D}_{K , I , J , P} + \\ {\sum }_{I = K + 1}^{N} {\sum }_{J = 1}^{m + 1} A {R}_{I , J} {\sum }_{P = 0}^{I - 1} F L {D}_{K , I , J , P} \\ = {\sum }_{J = 0}^{m + 1} \{ \begin{matrix}( K < N ? 1 \text{:} 0 ) A {L}_{K - 1 , J} \\ F {M}_{K - 1 , K + 1} A {R}_{K + 1 , J} + \\ {\sum }_{I = 1}^{K - 1} {\sum }_{P = K + 1}^{N} {\sum }_{Q = J + 1}^{m + 1} A {L}_{I , J} \\ F {A}_{I , J , P , Q} A {R}_{P , Q} / ( 1 - {P}_{d} )\end{matrix} \}\end{matrix} & ( 0.12 ) \\ where , & \\ F R {D}_{K , I , J , P} ≡ \{ \begin{matrix}F {R}_{I , J , P , P - 1} / ( 1 - {P}_{d} ) & if K < P - 1 \\ F {R}_{I , J , P , P - 2} & if K = P - 1 \\ 0 & if K = P \\ F {R}_{I , J , P , P - 1} & if K > P\end{matrix} & \\ F L {D}_{K , I , J , P} ≡ \{ \begin{matrix}F {L}_{I , J , P , P + 1} & if K < P \\ 0 & if K = P \\ F {L}_{I , J , P , P + 2} & if K = P + 1 \\ F {L}_{I , J , P , P + 1} / ( 1 - {P}_{d} ) & if K > P + 1\end{matrix} & \end{matrix}$$

As previously described, a small number (Δ≦8) of significant FRD or FLD terms in the inner summation. Also, only a banded region of width Δ≦8 of the arrays indexed by I and J is needed to be evaluated. Hence, the computation time of the first two summations in Equation (0.12) is approximately 2mΔ², while the time for the third summation in Equation (0.12) is approximately 2Δ⁴. This is an improvement over the original computation time of 2mΔ³, however the improvement can be greater if the equation is evaluated for all possible K(1≦K≦N), since in that case the innermost terms FA_{I,J,P,Q}, FR_{I,J,P,Q} and FL_{I,J,P,Q} for different K are similar and may be evaluated only once. For example, any term in the third summation is likely the same for all K s.t. I<K<P and absent for all other K. Thus all possible terms in the third summation can be computed in a single pass, and each term added to the probability sum of a number of results ƒ(D|H−H_{K}) for I<K<P. For each term, this can be done in constant time regardless of the range of possible K, an array of the differences of ƒ(D|H−H_{K}) is computed for consecutive K, and each term is add at the start of the K-range and subtract at the end of the K-range in the array of differences. From the array of differences, the individual ƒ(D|H−H_{K}) can be recovered at a later point. Similar argument applies to the terms in the first two summations of Equation (0.12), but each of the four variants involved should be computed and added to the corresponding four K ranges, which may only takes a constant amount of time. Thus, the overall time to evaluate ƒ(D|H−H_{K}) for all K is approximately 2mΔ³ plus the cost to pre-compute AL_{I,J} and AR_{I,J}, which are each also 2mΔ³. Thus, the total cost to compute all ƒ(D|H−H_{K}) is likely at most 3 times the cost to compute just AR_{I,J}, hence it is possible to compute ƒ(D|H−H_{K}) for all K for just 3 times, and the cost to compute it for a single K. If enough memory is available in the computer executing this process or other memory is available, the cost of computing the complex terms FA_{I,J,P,Q}, FR_{I,J,P,Q} and FL_{I,J,P,Q} can be shared between Equations (0.12)(0.5) and (0.3), which can reduce the total cost to perhaps just 2 times the cost to compute ƒ(D|H−H_{K}) for a single K.

The equivalent of the final Equation (0.12) for each of the remaining three local modifications to H is described below.

Equation (0.13) shows the result for adding a restriction site at H_{T} to H.

$$\begin{matrix}f ( D \vert H + {H}_{T} , {H}_{K - 1} < {H}_{T} < {H}_{K} ) = {\sum }_{I = 1}^{K - 1} {\sum }_{J = 0}^{m} {\sum }_{P = I + 1}^{N + 1} A {L}_{I , J} F R {A}_{K , I , J , P} + {\sum }_{I = K}^{N} {\sum }_{J = 1}^{m + 1} {\sum }_{P = 0}^{I - 1} A {R}_{I , J} F L {A}_{K , I , J , P} + {\sum }_{J = 0}^{m + 1} \{ \begin{matrix}A {L}_{K - 1 , J} F {M}_{K - 1 , K} A {R}_{K , J} + \\ {\sum }_{I = 1}^{K - 1} {\sum }_{P = K}^{N} {\sum }_{Q = J + 1}^{m + 1} A {L}_{I , J} F {A}_{I , J , P , Q} A {R}_{P , Q} ( 1 - {P}_{d} )\end{matrix} \} + {\sum }_{J = 0}^{m + 1} A L {T}_{J} A R {T}_{J} & ( 0.13 ) \\ where , & \\ A L {T}_{J} ≡ A {L}_{K , J} ( {H}_{K} -> {H}_{T} ) see recurrance for A {L}_{I , J} & \\ A R {T}_{J} ≡ A {R}_{K - 1 , J} ( {H}_{K - 1} -> {H}_{T} ) see recurrance for A {R}_{I , J} & \\ F R {A}_{K , I , J , P} ≡ \{ \begin{matrix}( 1 - {P}_{d} ) F {R}_{I , J , P , P - 1} & if K \leq P - 1 \\ F {R}_{I , J , K , K - 1} ( {H}_{K} -> {H}_{T} ) + F {R}_{I , J , K , K - 1} ( {H}_{K - 1} -> {H}_{T} ) & if K = P \\ F {R}_{I , J , P , P - 1} & if K > P\end{matrix} & \\ F L {A}_{K , I , J , P} ≡ \{ \begin{matrix}F {L}_{I , J , P , P + 1} & if K \leq P \\ F {K}_{I , J , K - 1 , K} ( {H}_{K - 1} -> {H}_{T} ) + F {L}_{I , J , K - 1 , K} ( {H}_{K} -> {H}_{T} ) & if K = P + 1 \\ ( 1 - {P}_{d} ) F {L}_{I , J , P , P + 1} & if K > P + 1\end{matrix} & \end{matrix}$$

The notation AL_{K,J}(H_{K}→H_{T}) means to evaluate AL_{K,J} (using its defining equation provided previously) while replacing any occurrence of H_{K} with H_{T}. Equation (0.13) preferably depends on the exact value of H_{T}, e.g., only in the last summation term. All other summations can be done simultaneously for all K (1≦K≦N+1) as described hereinabove for Equation (0.12). Only the last summation is preferably performed separately for each specific value of H_{T}. Hence, the total computation time is preferably proportional to 6mΔ³+4TΔ⁴, where T is the number of different values of H_{T} for which ƒ(D|H+H_{T}) is computed.

Equation (0.14) shows the result for increasing one fragment size between H_{K} and H_{K+1} by a specified amount Δ_{H}.

$$\begin{matrix}f ( D \vert H \text{:} {H}_{T} -> {H}_{T} + {Δ}_{H} ∀ T > K ) = {\sum }_{I = 1}^{K} {\sum }_{J = 0}^{m} {\sum }_{P = I + 1}^{N + 1} A {L}_{I , J} F R {D}_{K , I , J , P} + {\sum }_{I = K + 1}^{N} {\sum }_{J = 1}^{M + 1} {\sum }_{P = 0}^{I - 1} A {R}_{I , J} F L {D}_{K , I , J , P} + {\sum }_{J = 0}^{m + 1} \{ ( K \leq N ? 1 \text{:} 0 ) A {L}_{K , J} {P}_{v}^{{Δ}_{H}} F {M}_{K , K + 1} A {R}_{K + 1 , J} + {\sum }_{I = 1}^{K} {\sum }_{P = K + 1}^{N} {\sum }_{Q = J + 1}^{m + 1} A {L}_{I , J} F A {D}_{I , J , P , Q} A {R}_{P , Q} \} & ( 0.14 ) \\ where , & \\ F A {D}_{I , J , P , Q} ≡ F {A}_{I , J , P , Q} ( {H}_{P} -> {H}_{P} + {Δ}_{H} ) & \\ F R {D}_{K , I , J , P} ≡ \{ \begin{matrix}F {R}_{I , J , P , P - 1} ( {H}_{P - 1} -> {H}_{P - 1} + {Δ}_{H} , {H}_{P} -> {H}_{P} + {Δ}_{H} ) & if K < P - 1 \\ F {R}_{I , J , P , P - 1} ( {H}_{P} -> {H}_{P} + {Δ}_{H} ) & if K = P - 1 \\ F {R}_{I , J , P , P - 1} & if K \geq P\end{matrix} & \\ F L {D}_{K , I , J , P} ≡ \{ \begin{matrix}F {L}_{I , J , P , P + 1} & if K < P \\ F {L}_{I , J , P , P + 1} ( {H}_{P} -> {H}_{P} - {Δ}_{H} ) & if K = P \\ F {L}_{I , J , P , P + 1} ( {H}_{P} -> {H}_{P} - {Δ}_{H} , {H}_{P + 1} -> {H}_{P + 1} - {Δ}_{H} ) & if K > P\end{matrix} & \end{matrix}$$

If the same value of Δ_{H} is used for all fragments, it is possible to evaluate Equation (0.14) for all possible K (1≦K≦N+1) in time proportional to 6mΔ³, in the same manner as described above for Equation (0.12). On the other hand, if each Δ_{H} value is different, Equation (0.14) may be evaluated separately for each one at a cost proportional to 4mΔ²+2Δ⁴. To this result the costs of pre-computing AR_{I,J} and AL_{I,J} of 2mΔ³ should be added. Hence, the total cost for T unrelated Δ_{H} values can be proportional to 4mΔ³+T(4mΔ²+2Δ⁴). It may be possible to reduce this result to be to close to 4mΔ³+2TΔ⁴, since most of the terms in the first two summations in Equation (0.14) are likely to be negligible, except for a few terms when K is close to either end of H. This is the case for Equations (0.12) and (0.13) as well, while the cost of evaluating the terms of the first two summations are likely significant only in the current case, and even then likely only if m≧Δ².

In order to compute the first two (d=1,2) derivatives of ƒ(D|H) relative to all fragment sizes F_{K}≡H_{K+1}−H_{K},0≦K≦N, Equation is (0.15) can be used.

$$\frac{{\partial }^{d} f ( D \vert H )}{\partial {F}_{K}^{d}} = {\sum }_{I = 1}^{K} {\sum }_{J = 0}^{m} {\sum }_{P = I + 1}^{N + 1} A {L}_{I , J} \frac{{\partial }^{d} F {R}_{I , J , P , P - 1}}{\partial {F}_{K}^{d}} + {\sum }_{I = K + 1}^{N} {\sum }_{J = 1}^{m + 1} {\sum }_{P = 0}^{I - 1} A {R}_{I , J} \frac{{\partial }^{d} F {L}_{I , J , P , P + 1}}{\partial {F}_{K}^{d}} + ( K = N ? 1 \text{:} 0 ) A {L}_{N , M + I} \frac{{\partial }^{d} F M R}{\partial {F}_{N}^{d}} + ( K = 0 ? 1 \text{:} 0 ) A {R}_{1 , 0} \frac{{\partial }^{d} F M L}{\partial {F}_{0}^{d}} + {\sum }_{J = 0}^{m + 1} \{ \begin{matrix}( K < N ? 1 \text{:} 0 ) A {L}_{K , J} A {R}_{K + 1 , J} \frac{{\partial }^{d} F {M}_{K , K + 1}}{\partial {F}_{K}^{d}} + \\ {\sum }_{I = 1}^{K} {\sum }_{P = K + 1}^{N} {\sum }_{Q = J + 1}^{m + 1} A {L}_{I , J} A {R}_{P , Q} \frac{{\partial }^{d} F {A}_{I , J , P , Q}}{\partial {F}_{I}^{d}}\end{matrix} \} \qquad ( 0.15 )$$

The differential expressions in Equation (0.15) can be computed as shown in Equations (0.16)(0.17)(0.18)(0.19)(0.20)(0.21)(0.22) and (0.23).

$$\begin{matrix}\frac{{\partial }^{d} F {M}_{K , K + 1}}{\partial {F}_{K}^{d}} = F {{M}_{K , K + 1} ( log {P}_{v} )}^{d} & ( 0.16 ) \\ \frac{{\partial }^{d} F M R}{\partial {F}_{N}^{d}} = F {M}_{N , N + 1} ( {R}_{e} + log {P}_{v} ) {( log {P}_{v} )}^{d - 1} & \\ \frac{{\partial }^{d} F M L}{\partial {F}_{0}^{d}} = F {M}_{0 , 1} ( {R}_{e} + log {P}_{v} ) {( log {P}_{v} )}^{d - 1} & \\ \frac{\partial F {R}_{I , J , P , P - 1}}{\partial {F}_{K}} = \{ \begin{matrix}F R {A}_{I , J , P}^{′} - F R {B}_{I , J , P}^{′} & if K < P - 1 \\ F R {A}_{I , J , P}^{′} & if K = P - 1 \\ 0 & if K \geq P\end{matrix} & \\ \frac{\partial F {L}_{I , J , P , P - 1}}{\partial {F}_{K}} = \{ \begin{matrix}0 & if K < P \\ F L {A}_{I , J , P}^{′} & if K = P \\ F L {A}_{I , J , P}^{′} - F L {B}_{I , J , P}^{′} & if K > P\end{matrix} & \\ \frac{{\partial }^{2} F {R}_{I , J , P , P - 1}}{\partial {F}_{K}^{2}} = \{ \begin{matrix}F R {A}_{I , J , P}^{″} - F R {B}_{I , J , P}^{″} & if K < P - 1 \\ F R {A}_{I , J , P}^{″} & if K = P - 1 \\ 0 & if K \geq P\end{matrix} & \\ \frac{\partial F {L}_{I , J , P , P - 1}}{\partial {F}_{K}^{2}} = \{ \begin{matrix}0 & if K < P \\ F L {A}_{I , J , P}^{″} & if K = P \\ F L {A}_{I , J , P}^{″} - F L {B}_{I , J , P}^{″} & if K > P\end{matrix} & \\ \frac{\partial F {A}_{I , J , P , Q}}{\partial {F}_{I}} = F {A}_{I , J , P , Q} \{ \begin{matrix}\frac{{G}^{′} ( {D}_{Q} - {D}_{J} , {H}_{P} - {H}_{I} )}{G ( {D}_{Q} - {D}_{J} , {H}_{P} - {H}_{I} )} - \\ \frac{F {M}_{I , P} log {P}_{v}}{( 1 - F {M}_{I , P} )}\end{matrix} \} & ( 0.17 ) \\ \frac{{\partial }^{2} F {A}_{I , J , P , Q}}{\partial {F}_{I}^{2}} = F {A}_{I , J , P , Q} [ \begin{matrix}{\{ \begin{matrix}\frac{{G}^{′} ( {D}_{Q} - {D}_{J} , {H}_{P} - {H}_{I} )}{G ( {D}_{Q} - {D}_{J} , {H}_{P} - {H}_{I} )} - \\ \frac{F {M}_{I , P} log {P}_{v}}{( 1 - F {M}_{I , P} )}\end{matrix} \}}^{2} + \\ \begin{matrix}\frac{{G}^{″} ( {D}_{Q} - {D}_{J} , {H}_{P} - {H}_{I} )}{G ( {D}_{Q} - {D}_{J} , {H}_{P} - {H}_{I} )} - \\ {( \frac{{G}^{′} ( {D}_{Q} - {D}_{J} , {H}_{P} - {H}_{I} )}{G ( {D}_{Q} - {D}_{J} , {H}_{P} - {H}_{I} )} )}^{2} - \\ \frac{F {{M}_{I , P} ( log {P}_{v} )}^{2}}{{( 1 - F {M}_{I , P} )}^{2}}\end{matrix}\end{matrix} ] & \\ F R {A}_{I , J , P}^{′} ≡ {{λ}^{m - j} ( 1 - {P}_{d} )}^{P - I - 1} \{ \begin{matrix}( 1 - F {M}_{I , P} ) [ \begin{matrix}{R}_{e} {G}_{A} ( {D}_{m + 1} - {D}_{J} , {H}_{P} - {H}_{I} , \\ {H}_{P - 1} - {H}_{1} ) + ( P > N ? 1 \text{:} 0 ) \\ {G}^{′} ( {D}_{Q} - {D}_{J} , {H}_{P} - {H}_{I} )\end{matrix} ] \\ - F {M}_{I , P} ( log {P}_{v} ) [ \begin{matrix}{R}_{e} {G}_{E} ( {D}_{m + 1} - {D}_{J} , {H}_{P} - {H}_{I} , \\ {H}_{P - 1} - {H}_{1} ) + ( P > N ? 1 \text{:} 0 ) \\ {G}^{′} ( {D}_{Q} - {D}_{J} , {H}_{P} - {H}_{I} )\end{matrix} ]\end{matrix} \} & ( 0.18 ) \\ \begin{matrix}F R {B}_{I , J , P}^{′} ≡ {{λ}^{m - J} ( 1 - {P}_{d} )}^{P - I - 1} ( 1 - F {M}_{I , P} ) {R}_{e} {G}_{B} ( {D}_{m + 1} - {D}_{J} , \\ {H}_{P} - {H}_{I} , {H}_{P - 1} - {H}_{I} )\end{matrix} & \\ F L {A}_{I , J , P}^{′} ≡ {{λ}^{J - 1} ( 1 - {P}_{d} )}^{I - P - 1} \{ \begin{matrix}( 1 - F {M}_{P , I} ) [ \begin{matrix}{R}_{0} {G}_{A} ( {D}_{J ,} {H}_{I} - {H}_{P} , {H}_{I} - {H}_{P + 1} ) + \\ ( P = 0 ? 1 \text{:} 0 ) {G}^{′} ( {D}_{J} , {H}_{I} - {H}_{P} )\end{matrix} ] \\ - F {M}_{P , I} ( log {P}_{v} ) [ \begin{matrix}{R}_{e} {G}_{E} ( {D}_{J} , {H}_{I} - {H}_{P} , {H}_{I} - {H}_{P + 1} ) + \\ ( P = 0 ? 1 \text{:} 0 ) G ( {D}_{J} , {H}_{I} - {H}_{P} )\end{matrix} ]\end{matrix} \} & ( 0.19 ) \\ F L {B}_{I , J , P}^{′} ≡ {{λ}^{J - 1} ( 1 - {P}_{d} )}^{I - P - 1} ( 1 - F {M}_{P , I} ) {R}_{e} {G}_{B} ( {D}_{J} , {H}_{I} - {H}_{P} , {H}_{I} - {H}_{P + 1} ) & \\ F R {A}_{I , J , P}^{″} \approx {{λ}^{m - J} ( 1 - {P}_{d} )}^{P - I - 1} \{ \begin{matrix}{R}_{e} {G}_{A}^{′} ( {D}_{m + 1} - {D}_{J} , {H}_{P} - {H}_{I} , {H}_{P - 1} - {H}_{I} ) + \\ ( P > N ? 1 \text{:} 0 ) {G}^{′} ( {D}_{Q} - {D}_{J} , {H}_{P} - {H}_{I} )\end{matrix} \} & ( 0.20 ) \\ F R {B}_{I , J , P}^{″} \approx {{λ}^{m - J} ( 1 - {P}_{d} )}^{P - I - 1} {R}_{e} {G}_{B}^{′} ( {D}_{m + 1} - {D}_{J} , {H}_{P} - {H}_{I} , {H}_{P - 1} - {H}_{I} ) & \\ F L {A}_{I , J , P}^{″} ≡ {{λ}^{J - 1} ( 1 - {P}_{d} )}^{I - P - 1} \{ \begin{matrix}{R}_{e} {G}_{A}^{′} ( {D}_{J} , {H}_{I} - {H}_{P} , {H}_{I} - {H}_{P + 1} ) + \\ ( P = 0 ? 1 \text{:} 0 ) {G}^{″} ( {D}_{J} , {H}_{I} - {H}_{P} )\end{matrix} \} & ( 0.21 ) \\ F L {B}_{I , J , P}^{″} ≡ {{λ}^{J - 1} ( 1 - {P}_{d} )}^{I - P - 1} {R}_{e} {G}_{B}^{′} ( {D}_{J} , {H}_{I} - {H}_{P} , {H}_{I} - {H}_{P + 1} ) & \\ {G}_{A} ( d , {h}_{1} , {h}_{2} ) ≡ \frac{{e}^{- {( d - {h}_{1} )}^{2} / 2 {σ}^{2} A}}{\sqrt{2 π {σ}^{2} A}} , A \approx max ( min ( d , {h}_{1} ) , {h}_{2} ) & ( 0.22 ) \\ {G}_{B} ( d , {h}_{1} , {h}_{2} ) ≡ \frac{{e}^{- {( d - {h}_{2} )}^{2} / 2 {σ}^{2} A}}{\sqrt{2 π {σ}^{2} A}} \\ {G}_{A}^{′} ( d , {h}_{1} , {h}_{2} ) ≡ \frac{d - {h}_{1}}{{σ}^{2} A} {G}_{A} ( d , {h}_{1} , {h}_{2} ) \\ {G}_{B}^{′} ( d , {h}_{1} , {h}_{2} ) ≡ \frac{d - {h}_{2}}{{σ}^{2} A} {G}_{B} ( d , {h}_{1} , {h}_{2} ) & \\ G ( d , h ) ≡ \frac{{e}^{- {( d - h )}^{2} / 2 {σ}^{2} h}}{\sqrt{2 π {σ}^{2} h}} \\ {G}^{″} ( d , h ) ≡ [ {( \frac{{d}^{2} - {h}^{2} - {σ}^{2} h}{2 {σ}^{2} {h}^{2}} )}^{2} \frac{{d}^{2}}{{σ}^{2} {h}^{3}} + \frac{1}{2 {h}^{2}} ] G ( d , h ) \\ {G}^{″} ( d , h ) ≡ [ {( \frac{{d}^{2} - {h}^{2} - {σ}^{2} h}{2 {σ}^{2} {h}^{2}} )}^{2} \frac{{d}^{2}}{{σ}^{2} {h}^{3}} + \frac{1}{2 {h}^{2}} ] G ( d , h ) & ( 0.23 )\end{matrix}$$

### EXAMPLE 2

An application of one exemplary embodiment of the present invention to a simulated data set is described below. For this exemplary embodiment, the basic map assembly algorithms is preferably extended by adding a post processing phase to carefully examine the component input maps that go into each consensus map, assign each input map to one of two populations and reassemble them into two separate consensus maps. This implementation uses simulated data to allow the performance for data error rates greater than present in actual data to be determined.

To generate simulated data the first 5 megabases of human chromosome 21 published by NIH can be used, and an in-silico restriction map may be generated for the restriction enzyme PacI, and then random errors are repeatedly introduced into this restriction map using the error rates described above and selected a random piece of between 1.5 and 2.5 Megabases. This set of simulated data can represents one parental copy of chromosome 21. In order to generate the set for the other chromosome, the 5 Mb sequence can be randomly modified by inserting a random base modification to simulate SNPs and random insertions and deletions of about 3 Kb (the current sizing error averages 3 Kb per 30 Kb average restriction fragment, hence smaller insertions/deletions would likely be difficult to detect), so that the number of SNPs that coincide with restriction sites is approximately the same as the number of insertions and deletions. Such modified sequence can then be used to generate the second set of simulated maps, which correspond to the second parental copy of chromosome 21. The two sets of data may be combined in a 1:1 ratio and mixed together randomly.

The system, process and software arrangement according to the present invention can generate, e.g., 2 consensus maps and assign input maps to either of these consensus maps or can leave them unassigned. The accuracy of the results can be scored by comparing them with the true in-silico maps (generated along with the simulated data). This procedure can be repeated for different amounts of simulated data corresponding to data redundancy of 6×, 12×, 16×, 24× and 0×. Such redundancy can be measured per haplotype, and thus, the results for 6× redundancy generally corresponds to 6×2×5 Mb of simulated data or 30 molecules of average size 2 Mb. The exemplary results are summarized in Table 2. To further understand these results row 4 (16× Redundancy) can be reviewed. The last column shows that 80 molecules have been in the simulation. Of these molecules 71 molecules have been stated to be classified as belonging to one of the two phases (or haplotype variants). 2 errors were made and only 69 molecules have been correctly classified. By comparing the two consensus maps generated by the software, a list of restriction sites classified as polymorphic (i.e. a SNP was claimed by the software to exists at a restriction site), has been generated and this list was then compared to the correct list of SNPs generated from the true in-silico maps. The column with the header “fp SNPs” shows the number of generated false-positive SNPs (i.e. extra incorrect SNPs) and, in this case the number is 2. The column with the header “fn SNPs” shows the corresponding number of false-negative SNPs (i.e. SNPs missed by the software), and in this case the number is 1. Similarly for RFLPs (i.e. fragment size polymorphisms due to the simulated insertions/deletions), the numbers of false-positives is 0 and false-negatives is 12. The total numbers of correct SNPs and RFLPs are 16 and 24, respectively.

TABLE 2
Haplotyping algorithm performance for 16 SNPs and 24 RFLPs

| Redun- | | | | | Phase | |
|---|---|---|---|---|---|---|
| dancy | fp SNPs | fn SNPs | fp RFLPs | fn RFLPs | err | Molecules |
| 6x | 5 | 5 | 1 | 18 | 7/26 | 30 |
| 12x | 4 | 2 | 4 | 16 | 2/55 | 60 |
| 16x | 2 | 1 | 0 | 12 | 2/71 | 80 |
| 24x | 2 | 1 | 1 | 11 | 3/111 | 120 |
| 50x | 0 | 1 | 1 | 5 | 4/228 | 250 |
| 100x | 0 | 0 | 2 | 1 | 2/441 | 500 |

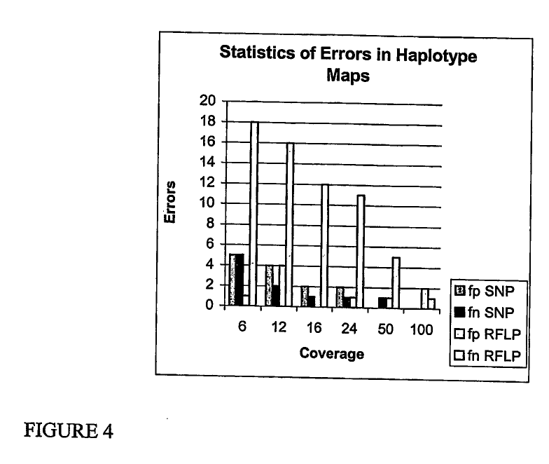

Exemplary statistics of errors in Haplotype maps is shown in FIG. 4. These exemplary results show the system process and software arrangement according to the present invention can advantageously classify molecules to the right phase (haplotype) whenever redundancy was 12× or higher. However, to detect all the SNPs and RFLPs in the data additional redundancy may be used. For example, at least 16-24× redundancy should be used to achieve 80% or more accuracy finding SNPs, and 50× redundancy to achieve similar accuracy finding RFLPs. These results indicate that with 50× data redundancy, it is possible to reliably detect most SNPs and over 80% of RFLPs for insertions/deletions equal to 1 standard deviation of the sizing error (currently 3 Kb). The accuracy for larger insertions/deletions would likely be higher.

Therefore, the exemplary embodiment of the system process and software arrangement according to the present invention is well-adapted to carry out the objects and attain the ends and advantages mentioned as well as those which are inherent therein. While the invention has been depicted, described, and is defined by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts and having the benefit of this disclosure. The depicted and described embodiments of the invention are exemplary only, and are not exhaustive of the scope of the invention. Consequently, the invention is intended to be limited only by the spirit and scope of the appended claims, giving full cognizance to equivalence in all respects.

1. A process for producing at least one genome wide map, comprising the steps of:
(a) preparing chromosome maps associated with at least one chromosome; and
(b) producing a portion of the at least one genome wide map based on the chromosome maps, wherein the at least one genome wide map comprises at least one of a haplotyped genome wide map or a genotyped genome wide map.

2-40. (canceled)

41. A software arrangement which, when executed on a processing device, configures the processing device to produce at least one genome wide map the software arrangement comprising:
(a) a first set of instructions which are capable of configuring the processing arrangement to prepare chromosome maps associated with at least one chromosome; and
(b) a first set of instructions which are capable of configuring the processing arrangement to produce a portion of the at least one genome wide map based on the chromosome maps, wherein the at least one genome wide map comprises at least one of a haplotyped genome wide map or a genotyped genome wide map.

42. The software arrangement according to claim 41, wherein the portion of at least one genome wide map comprises at least one restriction site.

43. The software arrangement according to claim 41, wherein less than all subparts of the genome wide map are produced in step (b) as ordered or unordered sets of contigs.

44. The software arrangement according to claim 41, wherein the chromosome maps are based on at least one single molecule map data set.

45. The software arrangement according to claim 41, wherein the genome wide map comprises two maps per chromosome is assembled from the at least one single molecule map data set

46. The software arrangement according to claim 44, wherein the at least one single molecule map data set has error rates as great as or smaller than: about 10% error in distance between sites, about 20% missing sites, about 7% false sites and about 50% of sites closer than about 1 kB apart that are indistinguishable.

47. The software arrangement according to claim 44, wherein the at least one single molecule map data set consists of either Optical Mapping data or any single molecule ordered maps of polymorphic markers comprising at least one of restriction site polymorphisms, restriction length polymorphisms, insertions of bases, deletions of bases, single nucleotide polymorphisms (SNPs).

48. The software arrangement according to claim 44, wherein the at least one single molecule map data sets comprising different restriction site markers are assembled into a single genome wide map wherein all restriction site markers are combined and wherein the restriction site markers can be distinguished.

49. The software arrangement according to claim 41, further comprising determining a conditional probability density expression.

50. The software arrangement according to claim 49, wherein the probability density expression is based on errors provided in at least one single molecule map data set.

51. The software arrangement according to claim 41, wherein substantially all site based polymorphisms are detected in the at least one genome wide map.

52. The software arrangement according to claim 41, wherein substantially all interval-based polymorphisms are detected in the at least one genome wide map.

53. The software arrangement according to claim 41, wherein steps (a) and (b) are performed within a particular time limit, and the particular time is a sub-quadratic function of a number of sites associated with an input data.

54. The software arrangement according to claim 41, further comprising performing a disease gene association study based on at least one genome wide map per patient.

55. A software arrangement which, when executed on a processing device, configures the processing device to perform disease gene association based on at least one haplotyped genome wide map per patient, the software arrangement comprising:
(a) a first set of instructions which are capable of configuring the processing arrangement to generate at least one haplotyped genome wide map per patient; and
(b) a second set of instructions which are capable of configuring the processing arrangement to perform the disease gene association based on the produced at least one haplotyped genome wide map.

56-82. (canceled)

83. A system for producing at least one genome wide map comprising a storage medium, wherein the storage medium includes software that is executed to perform the steps of:
(a) preparing chromosome maps associated with at least one chromosome; and
(b) producing a portion of the at least one genome wide map based on the chromosome maps, wherein the at least one genome wide map comprises at least one of a haplotyped genome wide map or a genotyped genome wide map.

84-122. (canceled)
